(19) United States
(12) Patent Application Publication
(10) Pub. No.: US 2004/0199018 A1
(43) Pub. Date: Oct. 7, 2004

(54) Bridged p-phenylenediamines

(76) Inventors: Georg Knuebel, Duesseldorf (DE); Bernd Meinigke, Leverkusen (DE); Horst Hoeffkes, Duesseldorf (DE); Helmut Giesa, Meerbusch (DE); David Rose, Hilden (DE)

Correspondence Address: HENKEL CORPORATION, THE TRIAD, SUITE 200, 2200 RENAISSANCE BLVD., GULPH MILLS, PA, 19406, US

(21) Appl. No.: 10/792,991

(22) Filed: Mar. 4, 2004

Related U.S. Application Data
(63) Continuation of application No. PCT/EP02/09875, filed on Sep. 4, 2002.

(30) Foreign Application Priority Data
Sep. 7, 2001 (DE) 101 44 226.2

(51) Int. Cl. C07C 215/28

(52) U.S. Cl. 564/443

(57) ABSTRACT

p-Phenylenediamines and derivatives thereof connected by polyoxaalkyl bridges to form dimers are highly suitable developer substances in oxidation hair colors. The bridged p-phenylenediamines are represented by the general formula (I)

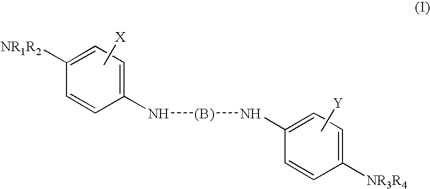

in which

(B) is a polyoxaalkyl bridge having at least two oxygen atoms;

X and Y independently of one another are H, Cl, F or a C₁-C₄ alkyl, aminoalkyl, alkoxy, C₂-C₄ dihydroxyalkyl, C₁-C₄ monohydroxyalkyl or C₂-C₄ alkenyl group; and

R1, R2, R3, and R4 independently of one another are H or a C₁-C₄ alkyl, C₁-C₄ monohydroxyalkyl or C₂-C₄ dihydroxyalkyl group, and acid addition salts derived therefrom.

A process for the preparation and a method of use of the bridged p-phenylenediamines of formula (I) are also disclosed.

### CROSS REFERENCE TO RELATED APPLICATIONS

This application is a continuation under 35 U.S.C. § 365(c) and 35 U.S.C. § 120 of international application PCT/EP02/09875, filed Sep. 4, 2002. This application also claims priority under 35 U.S.C. § 119 of DE 101 44 226.2, filed Sep. 7, 2001, which is incorporated herein by reference in its entirety.

### BACKGROUND OF THE INVENTION

The present invention relates to novel bridged p-phenylenediamines, to their use as a developer component in oxidation colorants, and to novel oxidation colorants which comprise the aforementioned bridged p-phenylenediamines. The present invention further relates to a process for preparing the bridged p-phenylenediamines.

For the coloring of keratinic fibers, especially human hair, a very wide variety of different colorants are known in the prior art. A preferred role in this context is played by what are called oxidation colorants, since they allow particularly intensive colorations and exhibit good fastness properties. The oxidation colorants comprise what are called developer components and coupler components as oxidation dye precursors. The developer components are also referred to as oxidation bases.

A wide variety of requirements are imposed on the oxidation dye precursors, and particularly on the developers:

on oxidative coupling they are required to develop the desired shades in sufficient intensity and naturalness.

they are required additionally to attach effectively to the fiber, and in particular

in the case of human hair there must be no marked differences apparent between exposed hair and new hair growth (leveling capacity).

they ought to be resistant to light, heat, perspiration, and the effect of chemical reducing agents, such as to perming fluids, for example.

moreover, when used as hair colorants they ought not to cause excessive staining of the scalp.

the oxidation dye products ought in particular to be absolutely unobjectionable from the toxicological and dermatological standpoints.

also desirable is an effective capacity for removal if the coloring results are unwanted.

Examples of customary coupler components include naphthols, resorcinol and resorcinol derivatives, pyrazolones, and m-aminophenols. Particularly suitable coupler substances include 1-naphthol, 1,5-, 2,7-, and 1,7-dihydroxynaphthalene, 5-amino-2-methylphenol, m-aminophenol, resorcinol, resorcinol monomethyl ether, m-phenylenediamine, 1-phenyl-3-methylpyrazol-5-one, 2,4-dichloro-3-aminophenol, 1,3-bis(2′,4′-diaminophenoxy)propane, 2-chlororesorcinol, 4-chloro-resorcinol, 2-chloro-6-methyl-3-aminophenol, 2-methyl-resorcinol, 5-methylresorcinol, and 2-methyl-4-chloro-5-aminophenol.

Developer components used frequently are primary aromatic amines having one further, free or substituted hydroxyl or amino group in para or ortho position, diaminopyridine derivatives, heterocyclic hydrazones, 4-aminopyrazolone derivatives, and 2,4,5,6-tetraaminopyrimidine and its derivatives. Widespread developer components also include p-phenylenediamine (PPD) and methyl-p-phenylenediamine, also referred to as p-tolylenediamine (PTD). These have been used for decades in permanent oxidation hair colors. In spite of their good performance properties, PPD and PTD cannot be used without reservation. Both PPD and PTD are not regarded as entirely unobjectionable from the toxicological standpoint.

A simple strategy for avoiding such toxicological problems lies in the synthesis of relatively large molecules, since for readily appreciable reasons these are less able to penetrate the skin and therefore are absorbed to a lesser degree if at all. In the synthesis and development of larger molecules, based for example on PPD units and PTD units, however, it must at the same time be borne in mind that PPD/PTD derivatives having very bulky substituents exhibit significantly poorer performance properties and are therefore suited only poorly, if at all, to use in oxidation colorants. In particular, with the aforementioned PPD/PTD-based developer substances having very bulky substituents, it is frequently possible only to obtain very poor color intensities.

In order to avoid the above-described toxicological problems, while taking account of the performance properties, proposals have been made in the prior art to link PPD or PTD units to form larger molecules, such as dimers. This has the advantage that not only are larger molecules obtained, but at the same time the number of reactive centers in the molecule is increased, thereby allowing an acceptable color intensity to be achieved. Linking can take place in a variety of ways and at different sites in the PPD or PTD units.

For example, in DE 2518393, tetraaminobiphenyls and tetraaminodiphenyl ethers are described as developer components. The aforementioned dimers are linked to one another via the benzene rings of the PPD units.

In the case of the PPD dimers described in DE 2852272 and DE 3149330 as well linking is directly via the benzene rings. The amino groups on the p-phenylenediamines therefore remain free. DE 2852272 and DE 3149330 propose bis(2,5-diaminophenoxy)alkanes as developer components.

While the majority of prior art PPD and/or PTD dimers, as described above, are linked directly via the benzene ring or rings of the PPD and PTD units, there are already a number of examples known of developer substances based on linking of PPD and/or PTD via the amino groups. In this context mention may be made of DE 19707545, which describes 1,4-diazacycloheptane derivatives as developer substances. In respect of the aforementioned N,N′-bis(4-aminophenyl)-1,4-diazacyclo-heptanes it is regarded as a disadvantage that the synthesis of such compounds is frequently accomplished only with a relatively high level of technical complexity, in an autoclave, for example.

It was an object of the present invention to provide novel developer components which particularly meet the requirements imposed on oxidation dye precursors. A particular object of the present invention was to find developer components which are based on p-phenylenediamine units and which on the basis of their molecular size can be regarded as toxicologically unobjectionable and at the same time lead to oxidative dyeings whose intensity is very good.

Furthermore, there is a continual need for new, improved dye components, since frequently with just one developer component or one specific coupler/developer combination it is impossible to obtain a shade which appears natural on the hair. In practice, therefore, it is usual to use combinations of different developer and/or coupler components.

It has now surprisingly been found that p-phenylenediamines and derivatives thereof connected by polyoxaalkyl bridges to form dimers are highly suitable developer substances in oxidation hair colors.

### DESCRIPTION OF THE INVENTION

The present invention accordingly first provides bridged p-phenylenediamines of the general formula (I)

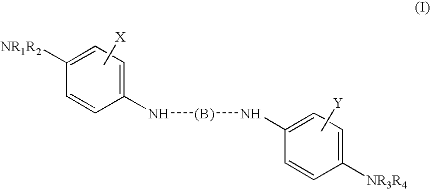

in which

(B) is a polyoxaalkyl bridge having at least two oxygen atoms;

X and Y independently of one another are H, Cl, F or a C₁-C₄ alkyl, aminoalkyl, alkoxy, C₂-C₄ dihydroxyalkyl, C₁-C₄ monohydroxyalkyl or C₂-C₄ alkenyl group; and

R1, R2, R3, and R4 independently of one another are H or a C₁-C₄ alkyl, C₁-C₄ monohydroxyalkyl or C₂-C₄ dihydroxyalkyl group, and acid addition salts derived therefrom.

In accordance with the present invention symmetrically substituted (R1=R3 and R2=R4) polyoxaalkyl-bridged p-phenylenediamines of the general formula (I) are preferred over corresponding asymmetrically substituted derivatives.

X and Y in the formula (I) in each case stand for one or more of the substituents indicated above. Accordingly the present invention also embraces bridged p-phenylenediamines which in addition to the two amino groups have one, two, three or even four substituents on the benzene ring of the phenylenediamine unit(s).

Examples of the C₁-C₄ alkyl groups specified as substituents in the compounds of the invention are the groups methyl, ethyl, propyl, isopropyl and/or butyl. Ethyl and/or methyl are preferred alkyl groups. A C₁-C₄ alkoxy group which is preferred in accordance with the invention is, for example, a methoxy and/or ethoxy group. In addition it is possible to specify, as preferred examples of a C₁-C₄ hydroxyalkyl group, a hydroxymethyl, 2-hydroxyethyl, 3-hydroxypropyl and/or 4-hydroxybutyl group. Particularly preferred C₂-C₄ dihydroxyalkyl groups are for example 1,2-dihydroxyethyl, 1,2-dihydroxypropyl, 1,3-dihydroxypropyl, 2,3-dihydroxypropyl, 1,2-dihydroxybutyl, 1,3-dihydroxybutyl, 1,4-dihydroxybutyl, 2,3-dihydroxybutyl, 2,4-dihydroxybutyl and/or 3,4-dihydroxybutyl groups. Particularly suitable C₂-C₄ alkenyl groups are the vinyl, allyl, 1-propenyl, 1-butenyl, 2-butenyl, 3-butenyl and/or 2-methylpropenyl group.

In one particularly preferred embodiment the present invention relates to bridged p-phenylenediamines having a structure in accordance with formula (II)

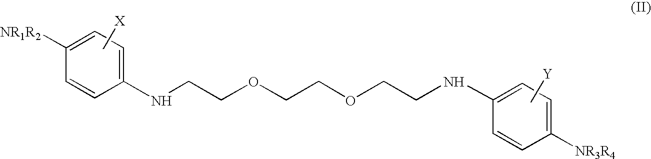

in which

X and Y independently of one another are H, Cl, F or a C₁-C₄ alkyl, aminoalkyl, alkoxy, C₂-C₄ dihydroxy-alkyl, C₁-C₄ monohydroxyalkyl or C₂-C₄ alkenyl group; and

R1, R2, R3, and R4 independently of one another are H or a C₁-C₄ alkyl, C₁-C₄ monohydroxyalkyl or C₂-C₄ dihydroxyalkyl group.

It is particularly preferred for X and Y and also the radicals R1, R2, R3, and R4 in the formula (II) to be H. The compound which results is 1,8-bis[(4-amino-phenyl)amino]-3,6-dioxaoctane.

In another particularly preferred embodiment the present invention relates to bridged p-phenylene-diamines having a structure in accordance with formula (III)

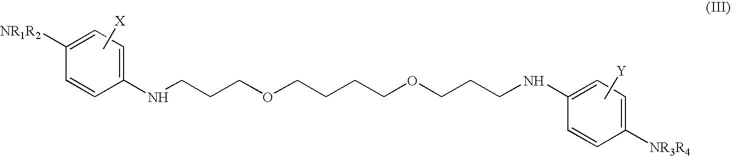

in which

X and Y independently of one another are H, Cl, F or a C₁-C₄ alkyl, aminoalkyl, alkoxy, C₂-C₄ dihydroxy-alkyl, C₁-C₄ monohydroxyalkyl or C₂-C₄ alkenyl group;, and

R1, R2, R3, and R4 independently of one another are H or a C₁-C₄ alkyl, C₁-C₄ monohydroxyalkyl or C₂-C₄ dihydroxyalkyl group.

It is particularly preferred for X and Y and also the radicals R1, R2, R3, and R4 in the formula (III) to be H. The compound which results is 1,12-bis[(4-amino-phenyl)amino]-4,9-dioxadodecane.

In yet another particularly preferred embodiment the present invention relates to bridged p-phenylenediamines having a structure in accordance with formula (IV)

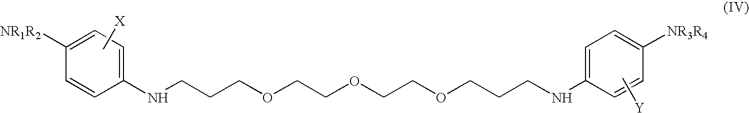

in which

X and Y independently of one another are H, Cl, F or a C₁-C₄ alkyl, aminoalkyl, alkoxy, C₂-C₄ dihydroxy-alkyl, C₁-C₄ monohydroxyalkyl or C₂-C₄ alkenyl group; and

R1, R2, R3, and R4 independently of one another are H or a C₁-C₄ alkyl, C₁-C₄ monohydroxyalkyl or C₂-C₄ dihydroxyalkyl group.

It is particularly preferred for X and Y and also the radicals R1, R2, R3, and R4 in the formula (III) to be H. The compound which results is 1,13-bis[(4-amino-phenyl)amino]-4,7,10-trioxatridecane.

The above-mentioned amino compounds of the invention can be converted readily into their corresponding acid addition salts. As well as the free bases, the present invention also relates to the acid addition salts derived from the respective compounds. Water-soluble, physiologically compatible salts which are suitable in accordance with the invention include, for example, the hydrochlorides, hydrobromides, sulfates, phosphates, acetates, propionates, citrates, and lactates. Particular preference is given to hydrochlorides.

In accordance with the present invention a process for preparing the bridged p-phenylenediamines of formulae (I), (II), (III) or (IV) according to the invention is provided which comprises the following steps:

a) reacting 1-fluoro-4-nitrobenzene with a polyoxa-alkyldiamine in the presence of a base, using 1-fluoro-4-nitrobenzene and the polyoxaalkyldiamine preferably in a ratio of 2:1, and

b) hydrogenating the intermediate obtained in step a).

Polyoxaalkyldiamines suitable in accordance with the invention include linear or branched polyoxaalkyls which have at least two, preferably primary amino groups. The polyoxaalkyl provided with at least two terminal amino functions forms the “bridge” provided in accordance with the invention in the resulting dimer. In the reaction of 1-fluoro-4-nitrobenzene with a polyoxaalkyldiamine in the presence of a base, the base used is preferably triethylamine or triethanolamine. It is preferred to employ the 1-fluoro-4-nitrobenzene, the polyoxaalkyldiamine and the base in a ratio of 2:1:2.

The reaction of 1-fluoro-4-nitrobenzene with polyoxoalkyldiamine in DMSO takes place preferably at elevated temperatures, preferably at a temperature of more than 50° C., more preferably at about 80° C.

It is particularly preferred in accordance with the invention for the polyoxoalkyldiamine to be reacted to be selected from the group consisting of 2,2′-(ethylenedioxy)diethylamine, 1,4-bis(3-aminopropoxy)butane, and 4,7,10-trioxa-1,13-tridecanediamine.

The bridged bis[(4-nitrophenyl)amino] compound obtained in step a) of the process of the invention is reduced in the following step b) to the corresponding bis[(4-aminophenyl)amino] compound. The nitro groups can be hydrogenated in accordance with methods known per se. Found suitable, for example, has been the catalytic hydrogenation with hydrogen in the presence of palladium on activated carbon (Pd/C).

In accordance with a further subject of the present invention an oxidation colorant for coloring keratinic fibers is provided which comprises as developer component at least one polyoxaalkyl-bridged p-phenylenediamine of the invention.

In one preferred embodiment of the present invention the oxidation colorant comprises at least one coupler component as well as the at least one developer component.

By keratin fibers or keratinic fibers for the purposes of the present invention are meant, according to the invention, furs, wool, feathers, hair, and in particular human hair. Although the oxidation colorants of the invention are suitable primarily for coloring keratin fibers, there is nothing in principle to prevent their use in other fields as well, particularly in color photography.

Particular preference is given for the purposes of the present invention to oxidation colorants in an aqueous vehicle or in powder form,

As coupler components it is possible in principle to use all of the coupler substances known in the prior art.

In one preferred embodiment of the present invention the oxidation colorant comprises at least one coupler component selected from the group consisting of m-phenylenediamine derivatives, naphthols, resorcinol and resorcinol derivatives, pyrazolones, and m-aminophenol derivatives.

Further coupler components preferred in accordance with the invention are

m-aminophenol and its derivatives such as for example 5-amino-2-methylphenol, 5-(β-hydroxyethyl)amino-2-methylphenol, N-cyclopentyl-3-aminophenol, 3-amino-2-chloro-6-methylphenol, 2-hydroxy-4-aminophenoxyethanol, 2,6-dimethyl-3-aminophenol, 3-trifluoroacetylamino-2-chloro-6-methylphenol, 5-amino-4-chloro-2-methylphenol, 5-amino-4-methoxy-2-methylphenol, 5-(2′-hydroxyethyl)amino-2-methylphenol, 3-(diethylamino)phenol, 1,3-dihydroxy-5-(methyl-amino)benzene, 3-ethylamino-4-methylphenol, and 2,4-dichloro-3-aminophenol,

o-aminophenol and its derivatives,

m-diaminobenzene and its derivatives such as for example 2,4-diaminophenoxyethanol, 1,3-bis(2′,4′-diaminophenoxy)propane, 1-methoxy-2-amino-4-(2′-hydroxyethylamino)benzene, 1, 3-bis(2′,4′-di-aminophenyl)propane, 2,6-bis(2′-hydroxyethylamino)-1-methylbenzene, 3,5-diamino-2-methoxytoluene, and 1-amino-3-bis(2′-hydroxyethyl)aminobenzene,

o-diaminobenzene and its derivatives such as for example 3,4-diaminobenzoic acid and 2,3-diamino-1-methylbenzene,

di- and trihydroxybenzene derivatives such as for example resorcinol, resorcinol monomethylether, 2-methylresorcinol, 5-methylresorcinol, 2,5-dimethylresorcinol, 2-chlororesorcinol, 4-chlororesorcinol, pyrogallol, and 1,2,4-trihydroxybenzene,

pyridine derivatives such as for example 2,6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine, and 3,5-diamino-2,6-dimethoxypyridine,

naphthalene derivatives such as for example 1-naphthol, 2-methyl-1-naphthol, 2-hydroxymethyl-1-naphthol, 2-hydroxyethyl-1-naphthol, 1,5-dihydroxynaphthalene, 1,6-dihydroxynaphthalene, 1,7-dihydroxy-naphthalene, 1,8-dihydroxynaphthalene, 2,7-dihydroxynaphthalene, and 2,3-dihydroxy-naphthalene,

morpholine derivatives such as for example 6-hydroxybenzomorpholine and 6-aminobenzomorpholine,

quinoxaline derivatives such as for example 6-methyl-1,2,3,4-tetrahydroquinoxaline,

pyrazole derivatives such as for example 1-phenyl-3-methylpyrazol-5-one,

indole derivatives such as for example 4-hydroxy-indole, 6-hydroxyindole, and 7-hydroxyindole,

pyrimidine derivatives, such as for example 4,6-diaminopyrimidine, 4-amino-2,6-dihydroxypyrimidine, 2,4-diamino-6-hydroxypyrimidine, 2,4,6-trihydroxypyrimidine, 2-amino-4-methylpyrimidine, 2-amino-4-hydroxy-6-methylpyrimidine, and 4,6-dihydroxy-2-methylpyrimidine, or

methylenedioxybenzene derivatives such as for example 1-hydroxy-3,4-methylenedioxybenzene, 1-amino-3,4-methylenedioxybenzene, and 1-(2′-hydroxyethyl)amino-3,4-methylenedioxybenzene.

Particularly suitable coupler substances include 1-naphthol, 1,5-, 2,7-, and 1,7-dihydroxynaphthalene, 5-amino-2-methylphenol, m-aminophenol, resorcinol, resorcinol monomethyl ether, m-phenylenediamine, 1-phenyl-3-methylpyrazol-5-one, 2,4-dichloro-3-aminophenol, 1, 3-bis(2′,4′-diaminophenoxy)propane, 2-chlororesorcinol, 4-chlororesorcinol, 2-chloro-6-methyl-3-aminophenol, 2-amino-3-hydroxypyridine, 2-methylresorcinol, 5-methylresorcinol, 3,5-diamino-2-methoxytoluene, 5-(β-hydroxyethyl)amino-2-methylphenol, and 2-methyl-4-chloro-5-aminophenol.

In another preferred embodiment the oxidation colorant of the invention includes, in addition to at least one polyoxaalkyl-bridged p-phenylenediamine of the invention, at least one further developer component, preferably selected from the group consisting of 2,4,5,6-tetraaminopyrimidine, p-aminophenol, N,N-bis(2′-hydroxyethyl)-p-phenylenediamine, 2-(2′,5′-diamino-phenyl)ethanol, 2-(2′,5′-diaminophenoxy)ethanol, 4-amino-3-methylphenol, 2-aminomethyl-4-aminophenol, 2-hydroxy-4,5,6-triaminopyrimidine, 2,4-dihydroxy-5,6-diamino-pyrimidine, 2,5,6-triamino-4-hydroxypyrimidine, 1,3-N,N′-bis(2′-hydroxyethyl)-N,N′-bis(4′-aminophenyl)diaminopropan-2-ol, 4-amino-2-((diethylamino)methyl)phenol, bis(2-hydroxy-5-aminophenyl)methane, and 4,5-diamino-1-(2′-hydroxyethylpyrazole) or salts thereof.

Additional developer components preferred in accordance with the invention further include p-phenylenediamine, p-tolylenediamine, p-aminophenol, o-aminophenol, 1-(2′-hydroxyethyl)-2,5-diaminobenzene, N,N-bis(2′-hydroxyethyl)-p-phenylenediamine, 2-(2′,5′-di-aminophenoxy)ethanol, 4-amino-3-methylphenol, 2,4,5,6-tetraaminopyrimidine, 2-hydroxy-4,5,6-triamino-pyrimidine, 4-hydroxy-2,5,6-triaminopyrimidine, 2,4-di-hydroxy-5,6-diaminopyrimidine, 2-dimethylamino-4,5,6-triaminopyrimidine, 2-hydroxymethylamino-4-amino-phenol, bis(4-aminophenyl)amine, 4-amino-3-fluorophenol, 2-aminomethyl-4-aminophenol, 2-hydroxymethyl-4-aminophenol, bis(2-hydroxy-5-aminophenyl)methane, 1,4-bis(4′-aminophenyl)diazacycloheptane, N,N′-bis-(2′-hydroxyethyl)-N,N′-bis(4′-aminophenyl)-1,3-diamino-propan-2-ol, 4-amino-2-(2′-hydroxyethoxy)phenol, 1,10-bis(2′,5′-diaminophenyl)-1,4,7,10-tetraoxadecane, and 4,5-diaminopyrazole derivatives in accordance with EP 0 740 931 or WO 94/08970 such as 4,5-diamino-1-(2′-hydroxyethyl)pyrazole, for example.

In accordance with the present invention it is envisaged that as well as the polyoxaalkyl-bridged p-phenylenediamine of the invention the hair colorant can include any desired further developer component.

It is preferred for the hair colorants of the invention to contain the developer components in an amount of from 0.005 to 20% by weight, preferably from 0.01 to 15% by weight, more preferably from 0.1 to 10% by weight, very preferably from 0.3 to 5% by weight, most preferably from 0.5 to 3% by weight, e.g., 1% and/or 2% by weight, and coupler components in an amount of from 0.005 to 20% by weight, preferably from 0.01 to 15% by weight, more preferably from 0.1 to 10% by weight, very preferably from 0.3 to 5% by weight, most preferably from 0.5 to 3% by weight, e.g., 1% and/or 2% by weight, based in each case on the total oxidation colorant.

Developer components and coupler components are generally employed in approximately equimolar amounts (taking into account the number of reactive centers in the respective components) to one another. Although equimolar use has proven advantageous, a certain excess of individual oxidation dye precursors is no disadvantage, and so developer components and coupler components may be present in a molar ratio of from 1:0.5 to 1:3, in particular from 1:1 to 1:2.

In a further embodiment preferred in accordance with the invention the colorant additionally comprises at least one direct dye.

Direct dyes are customarily nitrophenylenediamines, nitroaminophenols, azo dyes, anthraquinones or indophenols. Preferred direct dyes are the compounds known under the international designations or trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 12, Disperse Blue 3, HC Violet 1, Disperse Violet 1, Disperse Violet 4, Acid Violet 43, Disperse Black 9, and Acid Black 52, and also 1,4-diamino-2-nitrobenzene, 2-amino-4-nitrophenol, 1,4-bis(β-hydroxyethyl)amino-2-nitrobenzene, 3-nitro-4-(β-hydroxyethyl)aminophenol, 2-(2′-hydroxyethyl)amino-4,6-dinitrophenol, 1-(2′-hydroxyethyl)amino-4-methyl-2-nitrobenzene, 1-amino-4-(2′-hydroxyethyl)amino-5-chloro-2-nitrobenzene, 4-amino-3-nitrophenol, 1-(2′-ureidoethyl)amino-4-nitrobenzene, 4-amino-2-nitrodiphenylamine-2′-carboxylic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-1-hydroxy-4-nitrobenzene.

The compositions of the invention may further comprise a cationic direct dye. Particular preference is given in this context to

(a) cationic triphenylmethane dyes, such as for example Basic Blue 7, Basic Blue 26, Basic Violet 2, and Basic Violet 14,

(b) aromatic systems substituted by a quaternary nitrogen group, such as for example Basic Yellow 57, Basic Red 76, Basic Blue 99, Basic Brown 16, and Basic Brown 17, and

(c) direct dyes containing a heterocycle having at least one quaternary nitrogen atom, as specified for example in EP-A2-998 908, expressly incorporated here by reference, in claims 6 to 11.

Preferred cationic direct dyes of group (c) are in particular the following compounds:

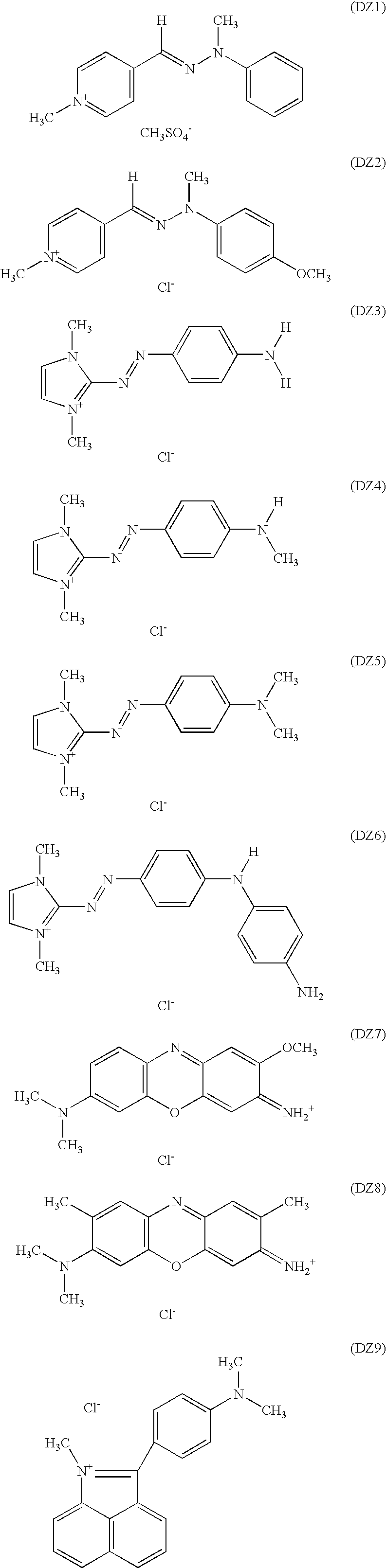

The compounds of the formulae (DZ1), (DZ3), and (DZ5) are especially preferred cationic direct dyes of group (c). The cationic direct dyes sold under the trade name Arianor® are direct dyes which are likewise particularly preferred in accordance with the invention.

The compositions of the invention in accordance with this embodiment contain the direct dyes preferably in an amount of from 0.01 to 20% by weight, based on the total colorant.

The compositions of the invention may further comprise naturally occurring dyes such as for example henna red, henna neutral, henna black, chamomile blossom, sandalwood, black tea, buckthorn bark, sage, logwood, madder root, catechu, cedar, and alkanna root.

The compositions of the invention contain the direct dyes preferably in an amount of from 0.005 to 20% by weight, preferably from 0.01 to 15% by weight, more preferably from 0.1 to 10% by weight, very preferably from 0.3 to 5% by weight, most preferably from 0.5 to 3% by weight, e.g., 1% and/or 2% by weight, based on the total oxidation colorant.

It is not required that the oxidation dye precursors or the direct dyes each constitute unitary compounds. Rather, within the hair colorants of the invention, owing to the preparation processes for the individual dyes, there may also be minor amounts of further components, provided they do not adversely affect the coloring result or require exclusion on other grounds, e.g., toxicological grounds.

With regard to the dyes which can be used in the hair colorants and tinting compositions of the invention reference is further made expressly to the monograph by Ch. Zviak, The Science of Hair Care, chapter 7 (pages 248-250; direct dyes) and also chapter 8, pages 264-267; oxidation dye precursors), published as volume 7 in the series “Dermatology” (eds.: Ch. Culnan and H. Maibach), Marcel Dekker Inc., New York, Basle, 1986, and also to the “European inventory of cosmetic raw materials”, published by the European Community, obtainable in disk form from the Bundesverband Deutscher Industrie-und Handelsunternehmen für Arzneimittel, Reformwaren und Körperpflegemittel e.V., Mannheim.

For preparing the colorants of the invention the oxidation dye precursors can be incorporated into a cosmetically appropriate vehicle—a hydrous vehicle, for example. For the purposes of hair coloring, such vehicles are for example creams, emulsions, gels or else foaming surfactant solutions, e.g., shampoos, foam aerosols or other formulations suitable for use on hair.

The colorants of the invention may further comprise all actives, additives, and auxiliaries that are known for such formulations. In many cases the colorants include at least one surfactant, with in principle not only anionic but also zwitterionic, ampholytic, nonionic, and cationic surfactants being suitable. In many cases it has, however, proven advantageous to select the surfactants from anionic, zwitterionic or nonionic surfactants.

The compositions of the invention preferably contain surfactants in an amount of from 0.1 to 30% by weight, preferably from 1 to 20% by weight, and more preferably from 2 to 10% by weight.

Anionic surfactants suitable in formulations of the invention include all anionic surface-active substances suitable for use on the human body. They are characterized by a water-solubilizing, anionic group such as, for example, a carboxylate, sulfate, sulfonate or phosphate group and a lipophilic alkyl group having about 10 to 22 carbon atoms. The molecule may additionally contain glycol or polyglycol ether groups, ester, ether, and amide groups, and also hydroxyl groups. Examples of suitable anionic surfactants, in each case in the form of the sodium, potassium, and ammonium salts and of the mono-, di-, and trialkanolammonium salts with 2 or 3 carbon atoms in the alkanol group, are

linear and/or branched fatty acids having 8 to 22 carbon atoms (soaps),

ether carboxylic acids of formula R—O—(CH₂—CH₂O)ₓ—CH₂—COOH in which R is a linear alkyl group having 10 to 22 carbon atoms and x=0 or is from 1 to 16,

acylsarcosides having 10 to 18 carbon atoms in the acyl group,

acyltaurides having 10 to 18 carbon atoms in the acyl group,

acylisethionates having 10 to 18 carbon atoms in the acyl group,

sulfosuccinic monoalkyl and dialkyl esters having 8 to 18 carbon atoms in the alkyl group and sulfosuccinic monoalkyl polyoxyethyl esters having 8 to 18 carbon atoms in the alkyl group and 1 to 6 oxyethyl groups,

linear alkanesulfonates having 12 to 18 carbon atoms,

linear alpha-olefinsulfonates having 12 to 18 carbon atoms,

alpha-sulfo fatty acid methyl esters of fatty acids having 12 to 18 carbon atoms,

alkyl sulfates and alkyl polyglycol ether sulfates of the formula R—O(CH₂—CH₂O)ₓ—SO₃H in which R is a preferably linear alkyl group having 10 to 18 carbon atoms and x=0 or is from 1 to 12,

mixtures of surface-active hydroxysulfonates in accordance with DE-A-37 25 030,

sulfated hydroxyalkylpolyethylene and/or hydroxy-alkylenepropylene glycol ethers in accordance with DE-A-37 23 354,

sulfonated unsaturated fatty acids having 12 to 24 carbon atoms and from 1 to 6 double bonds in accordance with DE-A-39 26 344,

esters of tartaric acid and citric acid with alcohols, which represent adducts of about 2-15 molecules of ethylene oxide and/or propylene oxide with fatty alcohols having 8 to 22 carbon atoms.

Preferred anionic surfactants are alkyl sulfates, alkyl polyglycol ether sulfates, and ether carboxylic acids having 10 to 18 carbon atoms in the alkyl group and up to 12 glycol ether groups in the molecule, and also, in particular, salts of saturated and in particular unsaturated C₈-C₂₂ carboxylic acids, such as oleic acid, stearic acid, isostearic acid, and palmitic acid.

The compositions of the invention may contain anionic surfactants preferably in an amount of from 0.5 to 30% by weight, more preferably from 1 to 20% by weight.

Surfactants termed zwitterionic surfactants are those surface-active compounds which in the molecule carry at least one quaternary ammonium group and at least one —COO⁽⁻⁾ or —SO₃⁽⁻⁾ group. Particularly suitable zwitterionic surfactants are those known as betaines, such as the N-alkyl-N,N-dimethylammonium glycinates, cocoalkyl-dimethylammonium glycinate for example, N-acyl-aminopropyl-N,N-dimethylammonium glycinates, cocoacylaminopropyl-dimethylammonium glycinate for example, and 2-alkyl-3-carboxymethyl-3-hydroxyethyl-imidazolines having in each case 8 to 18 carbon atoms in the alkyl or acyl group, and also cocoacylaminoethylhydroxyethylcarboxymethyl glycinate. A preferred zwitterionic surfactant is the fatty acid amide derivative known under the INCI name Cocamidopropyl Betaine.

The compositions of the invention may contain zwitterionic surfactants preferably in an amount of from 0.1 to 20% by weight, more preferably from 1 to 10% by weight.

By ampholytic surfactants are meant those surface-active compounds which in addition to a C₈₋₁₈ alkyl or acyl group in the molecule contain at least one free amino group and at least one —COOH or —SO₃H group and are capable of forming inner salts. Examples of suitable ampholytic surfactants are N-alkylglycines, N-alkylpropionic acids, N-alkylaminobutyric acids, N-alkyliminodipropionic acids, N-hydroxyethyl-N-alkyl-amidopropylglycines, N-alkyltaurines, N-alkylsarcosines, 2-alkylaminopropionic acids, and alkylaminoacetic acids, having in each case about 8 to 18 carbon atoms in the alkyl group. Particularly preferred ampholytic surfactants are N-cocoalkylaminopropionate, cocoacylaminoethylamino-propionate, and C₁₂₋₁₈ acylsarcosine.

The compositions of the invention may contain ampholytic surfactants preferably in an amount of from 0.1 to 20% by weight, more preferably from 1 to 10% by weight,

Nonionic surfactants include as hydrophilic group, for example, a polyol group, a polyalkylene glycol ether group or a combination of polyol and polyglycol ether group. Examples of such compounds include

adducts of from 2 to 30 mol of ethylene oxide and/or from 0 to 5 mol of propylene oxide with linear fatty alcohols having 8 to 22 carbon atoms, with fatty acids having 12 to 22 carbon atoms, and with alkylphenols having 8 to 15 carbon atoms in the alkyl group,

C₁₂₋₂₂ fatty acid monoesters and diesters of adducts of from 1 to 30 mol of ethylene oxide with glycerol,

C₈₋₂₂ alkylmonoglycosides and alkyloligoglycosides, their ethoxylated analogs and/or their esters, with tartaric and/or citric acid, for example,

adducts of from 5 to 60 mol of ethylene oxide with castor oil and hydrogenated castor oil,

adducts of ethylene oxide with sorbitan fatty acid esters, and

adducts of ethylene oxide with fatty acid alkanol amides.

The compositions of the invention may contain nonionic surfactants preferably in an amount of from 0.1 to 30% by weight, more preferably from 1 to 20% by weight.

Examples of the cationic surfactants which can be used in the hair treatment compositions of the invention are in particular quaternary ammonium compounds. Preference is given to ammonium halides such as alkyltrimethylammonium chlorides, dialkyldimethylammonium chlorides, and trialkylmethylammonium chlorides, e.g., cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride. Further cationic surfactants which can be used in accordance with the invention are the quaternized protein hydrolysates.

Likewise suitable in accordance with the invention are cationic silicone oils such as, for example, the commercially available products Q2-7224 (manufacturer: Dow Corning; a stabilized trimethylsilylamodimethicone), Dow Corning 929 emulsion (containing a hydroxyl-amino-modified silicone, also referred to as Amodimethicone), SM-2059 (manufacturer: General Electric), SLM-55067 (manufacturer: Wacker), and Abil®-Quat 3270 and 3272 (manufacturer: Th. Goldschmidt; diquaternary polydimethylsiloxanes, Quaternium-80).

Alkylamidoamines, particularly fatty acid amidoamines such as the stearylamidopropyldimethylamine obtainable under the designation Tego Amid® S 18, are distinguished not only by good conditioning but also, specifically, by their ready biodegradability. Likewise biodegradable are quaternary ester compounds, called ester quats, such as the methylhydroxyalkyl-dialkoyloxyalkylammonium methosulfates sold under the trademark Stepantex®, and the products sold under the trade name Dehyquart® such as Dehyquart® AU-46.

One example of a quaternary sugar derivative which can be used as a cationic surfactant is the commercial product Glucquat® 100, according to INCI nomenclature a Lauryl Methyl Gluceth-10 Hydroxypropyl Dimonium Chloride.

The compositions of the invention may comprise cationic surfactants preferably in an amount of from 0.1 to 20% by weight, more preferably from 1 to 10% by weight.

In each case of the compounds containing alkyl groups that are employed as surfactants the substances in question can be unitary substances. It is generally preferred, however, to start from natural plant or animal raw materials in the preparation of these substances, so giving substance mixtures having different alkyl chain lengths depending on the particular raw material.

In the case of the surfactants which represent adducts of ethylene oxide and/or propylene oxide with fatty alcohols, or derivatives of these adducts, it is possible to use products having a “normal” homolog distribution and products having a narrowed homolog distribution. By “normal” homolog distribution are meant mixtures of homologs which are obtained in the reaction of fatty alcohol and alkylene oxide using alkali metals, alkali metal hydroxides or alkali metal alkoxides as catalysts. Narrowed homolog distributions, in contrast, are obtained when using, for example, hydrotalcites, alkaline earth metal salts of ether carboxylic acids, alkaline earth metal oxides, hydroxides or alkoxides as catalysts. The use of products having a narrowed homolog distribution can be preferable.

In accordance with the invention the use of anionic surfactants in combination with zwitterionic surfactants can be particularly preferable.

Likewise preferred in accordance with the invention are those compositions further comprising a cationic polymer.

Among the cationic polymers preference is given to the permanently cationic polymers. Polymers are referred to as “permanently cationic” in accordance with the invention if they contain a cationic group independently of the pH of the composition.

These in general are polymers containing a quaternary nitrogen atom, in the form for example of an ammonium group.

Examples of preferred cationic polymers include the following:

quaternized cellulose derivatives as obtainable under the names Celquat® and Polymer JR® commercially. The compounds Celquat® H 100, Celquat® L 200, and Polymer JR® 400 are preferred quaternized cellulose derivatives;

polysiloxanes having quaternary groups, such as, for example, the commercially available products Q2-7224 (manufacturer: Dow Corning; a stabilized trimethylsilylamodimethicone), Dow Corning® 929 emulsion (containing a hydroxyl-amino-modified silicone, also referred to as Amodimethicone), SM-2059 (manufacturer: General Electric), SLM-55067 (manufacturer: Wacker), and Abil®-Quat 3270 and 3272 (manufacturer: Th. Goldschmidt;

diquaternary polydimethylsiloxanes, Quaternium-80);

cationic guar derivatives, such as particularly the products sold under the trade names Cosmedia® Guar and Jaguar®;

polymeric dimethyldiallylammonium salts and copolymers thereof with esters and amides of acrylic and methacrylic acid. The products available commercially under the names Merquat® 100 (poly(dimethyldiallylammonium chloride)) and Merquat® 550 (dimethyldiallylammonium chloride-acrylamide copolymer) are examples of such cationic polymers;

copolymers of vinylpyrrolidone with quaternized derivatives of dialkylaminoacrylate and -meth-acrylate, such as, for example, vinylpyrrolidone-dimethylaminomethacrylate copolymers quaternized with diethyl sulfate. Such compounds are available commercially under the names Gafquat® 734 and Gafquat® 755;

vinylpyrrolidone-vinylimidazolium methochloride copolymers, as sold under the names Luviquat® FC 370, FC 550, FC 905, and HM 552.

quaternized polyvinyl alcohol,

and also the polymers known under the names

Polyquaternium 2,

Polyquaternium 17,

Polyquaternium 18, and

Polyquaternium 27, having quaternary nitrogen atoms in the polymer main chain.

Likewise as cationic polymer use may be made of the polymers known under the names Polyquaternium-24 (commercial product, e.g., Quatrisoft® LM 200), Polyquaternium-32, Polyquaternium-35, and Polyquaternium-37 (commercial products, e.g., Salcare® SC 92 and Salcare® SC 95). Likewise possible for use in accordance with the invention are the copolymers of vinylpyrrolidone such as are obtainable as commercial products Copolymer 845 (manufacturer: ISP), Gaffix® VC 713 (manufacturer: ISP), Gafquat® ASCP 1011, Gafquat® HS 110, Luviquat® 8155, and Luviquat® MS 370.

Cationic polymers preferred in accordance with the invention are quaternized cellulose derivatives, polymeric dimethyldiallylammonium salts, Polyquaternium-27 and copolymers thereof, and also polymers of the Polyquaternium-2 type, Cationic cellulose derivatives, particularly the commercial product Polymer® JR400, and polymers of the Polyquaternium-2 type, particularly the commercial product Mirapol® A-15, are especially preferred cationic polymers.

The compositions of the invention may contain cationic polymers preferably in an amount of from 0.1 to 10% by weight, more preferably from 0.2 to 5% by weight.

In many cases it is also possible, as an alternative to the cationic polymers, to use amphopolymers. The generic term amphopolymers embraces amphoteric polymers, i.e., polymers which contain both free amino groups and free —COOH or SO₃H groups in the molecule and are capable of forming inner salts, zwitterionic polymers, which contain in the molecule quaternary ammonium groups and —COO⁻ or —SO₃⁻ groups, and those polymers which contain —COOH or SO₃H groups and quaternary ammonium groups. An example of an amphopolymer which can be used in accordance with the invention is the acrylic resin obtainable under the name Amphomer®, which constitutes a copolymer of tert-butylaminoethyl methacrylate, N-(1,1,3,3-tetramethylbutyl)acrylamide, and two or more monomers from the group consisting of acrylic acid, methacrylic acid, and the simple esters thereof. Likewise preferred amphopolymers are composed of unsaturated carboxylic acid (e.g., acrylic and methacrylic acid), cationically derivatized unsaturated carboxylic acid (e.g., acrylamidopropyltrimethylammonium chloride), and, if desired, further ionic or nonionogenic monomers, as may be taken, for example, from German laid-open specification 39 29 973 and the prior art cited therein.

It is likewise possible in accordance with the invention to employ terpolymers of acrylic acid, methyl acrylate, and methacrylamidopropyltriammonium chloride, such as are obtainable commercially under the name Merquat® 2001 N, and also the commercial product Merquat® 280. Particularly preferred polymers are, for example, acrylamidopropyltrimethylammonium chloride/acrylate copolymers and/or octylacrylamide/methyl methacrylate/tert-butylaminoethyl methacrylate/2-hydroxypropyl methacrylate copolymers.

The compositions of the invention may contain amphopolymers preferably in an amount of from 0.1 to 10% by weight.

Likewise with preference the compositions of the invention comprise at least one nonionogenic or anionic polymer having thickening properties. Preference is given in this context to (optionally crosslinked) polyacrylic acids, cellulose derivatives, e.g. methyl-cellulose, hydroxyalkylcellulose, and carboxymethyl-cellulose, and xanthah gum.

The compositions of the invention may contain nonionogenic and/or anionic polymers preferably in an amount of from 0.1 to 25% by weight.

Further actives, auxiliaries, and additives are for example:

thickeners such as agar agar, guar gum, alginates, xanthan gum, gum arabic, karaya gum, carob gum, linseed gums, dextrans, cellulose derivatives, e.g., methylcellulose, hydroxyalkylcellulose, and carboxymethylcellulose, starch fractions and derivatives such as amylose, amylopectin, and dextrins, clays such as bentonite, for example, or entirely synthetic hydrocolloids such as polyvinyl alcohol, for example;

structurants such as glucose and maleic acid;

hair-conditioning compounds such as phospholipids, e.g., soya lecithin, egg lecithin, and cephalins, and also silicone oils;

protein hydrolysates, especially elastin, collagen, keratin, milk protein, soya protein, and wheat protein hydrolysates, their condensation products with fatty acids, and quaternized protein hydrolysates;

perfume oils, dimethylisosorbide, and cyclodextrins;

solubilizers such as ethanol, isopropanol, ethylene glycol, propylene glycol, glycerol, and diethylene glycol;

antidandruff actives such as piroctone olamine and zinc omadine;

further substances for adjusting the pH;

actives such as panthenol, pantothenic acid, allantoin, pyrrolidonecarboxylic acids and salts thereof, plant extracts, and vitamins;

cholesterol;

light stabilizers;

consistency agents such as sugar esters, polyol esters or polyol alkyl ethers;

fats and waxes such as spermaceti, beeswax, montan wax, paraffins, fatty alcohols, and fatty acid esters;

fatty acid alkanol amides;

complexing agents such as EDTA, NTA, and phosphonic acids,

swelling agents and penetrants such as glycerol, propylene glycol monoethyl ether, carbonates, hydrogen carbonates, guanidines, ureas, and primary, secondary and tertiary phosphates;

opacifiers such as latex;

pearlescents such as ethylene glycol monostearate and distearate;

propellants such as propane/butane mixtures, N₂O, dimethyl ether, CO₂ and air; and

antioxidants.

The constituents of the hydrous vehicle are used, for preparing the colorants of the invention, in amounts customary for this purpose: for example, emulsifiers are used in concentrations of from 0.1 to 30% by weight, preferably from 0.2 to 10% by weight, more preferably from 0.3 to 5% by weight, most preferably from 0.5 to 3% by weight, and thickeners in concentrations of from 0.1 to 25% by weight, preferably from 0.2 to 10% by weight, more preferably from 0.3 to 5% by weight, most preferably from 0.5 to 2% by weight, based in each case on the total amount of the colorant.

The oxidative development of the coloration can in principle take place with atmospheric oxygen. It is also possible, however, to use a chemical oxidant. It is especially preferred when on human hair the desire is not only for coloring but also for a lightening effect. Suitable oxidants include persulfates, chlorites, and, in particular, hydrogen peroxide or its adducts with urea, melamine, and sodium borate. A further possibility is to carry out the oxidation using enzymes. The enzymes in this case (enzyme class 1: oxidoreductases) may transfer electrons from suitable developer components (reductants) to atmospheric oxygen. Preference is given to oxidases such as tyrosinase and laccase, but also to glucose oxidase, uricase or pyruvate oxidase. It is additionally possible for the enzymes to serve for reinforcing the action of small amounts of oxidants that are present. One example of an enzymatic method of this kind is the approach which involves reinforcing the action of small amounts (e.g., 1% or less, based on the total composition) of hydrogen peroxide by means of peroxidases.

Advantageously the oxidant formulation is mixed immediately before hair coloring with the formulation comprising the oxidation dye precursors. The resultant ready-to-use hair coloring product ought to have a pH in the range from 6 to 13, preferably in a range from 8 to 12, most preferably in a range from 9 to 11. Particular preference is given to employing the hair colorants in a weakly alkaline medium. The application temperatures can be situated within a range between 15 and 40° C. After an exposure period of approximately 30 minutes the hair colorant is removed by rinsing from the hair that is to be colored. Subsequent shampooing is unnecessary if a vehicle with a high surfactant content, e.g., a coloring shampoo, has been used.

A further subject of this invention lies in the use of the aforementioned compositions for coloring keratinic fibers.

The examples which follow are intended to illustrate the subject-matter of the invention, but should not be understood as constituting any restriction thereon.

### EXAMPLES

### a) Preparing the Compounds

### 1. 1,8-bis[(4-Aminophenyl)amino]-3,6-dioxaoctane×4 HCl (1)

1.1 1,8-bis[(4-Nitrophenyl)amino]-3,6-dioxaoctane 100 ml of DMSO were charged to a 500 ml stirred apparatus and 10.3 g of triethylamine, 7.6 g of 2,2′-(ethylenedioxy)diethylamine, and 14.4 g of 1-fluoro-4-nitrobenzene were added, The mixture was stirred at 80° C. for 16 h and after cooling was poured onto 1 l of ice-water. The precipitate formed was filtered off with suction and washed with water. This was followed by drying at 40° C. under reduced pressure.

Yield: 18.5 g (94%) m.p.: 130-133° C.

### 1.2 1,8-bis[(4-Aminophenyl)amino]-3,6-dioxaoctane

To 17.6 g of 1,8-bis[(4-nitrophenyl)amino]-3,5-dioxaoctane in 400 ml of ethanol there were added 1.5 g of Pd/C (5%). Catalytic reduction with hydrogen was carried out, while shaking. After the end of hydrogen absorption 100 ml of dilute HCl were added, the mixture was filtered, and the filtrate was evaporated to dryness and dried at 50° C. under reduced pressure.

Yield: 22.2 g (103%) m.p.: 183-225° C. (decomposition)

### 2. 1,12-bis[(4-Aminophenyl)amino]-4,9-dioxadodecane×4 HCl (2)

2.1 1,12-bis[(4-Nitrophenyl)amino]-4,9-dioxadodecane 100 ml of DMSO were charged to a 500 ml stirred apparatus and 10.3 g of triethylamine, 10.5 g of 1,4-bis(3-aminopropoxy)butane, and 14.4 g of 1-fluoro-4-nitrobenzene were added. The mixture was stirred at 80° C. for 20 h and after cooling was poured onto 1 l of ice-water. The precipitate formed was filtered off with suction and washed with water. This was followed by drying at 50° C. under reduced pressure.

Yield: 21.3 g (95%) m.p.: 112-115° C.

### 2.2 1,12-bis[(4-Aminophenyl)amino]-4,9-dioxadodecane×4 HCl

To 20.1 g of 1,12-bis[(4-nitrophenyl)amino]-4,9-dioxa-dodecane in 400 ml of ethanol there were added 1.2 g of Pd/C (5%). Catalytic reduction with hydrogen was carried out, while shaking. After the end of hydrogen absorption 100 ml of dilute HCl were added, the mixture was filtered, and the filtrate was evaporated to dryness and dried at 50° C. under reduced pressure.

Yield: 20.2 g (84%) m.p.: 180° C. (decomposition)

### 3. 1,13-bis[(4-Aminophenyl)amino]-4,7,10-trioxatridecane×4 HCl (3)

3.1 1,13-bis[(4-Nitrophenyl)amino]-4,7,10-trioxatridecane 100 ml of DMSO were charged to a 500 ml stirred apparatus and 10.3 g of triethylamine, 11.2 g of 4,7,10-trioxa-1,13-tridecanediamine, and 14.4 g of 1-fluoro-4-nitrobenzene were added. After 19 h of stirring at 80° C. the reaction mixture was cooled and then poured onto 1 l of ice-water. The water was decanted from the resultant oily precipitate, a further 1 l of water was added, and the mixture was stirred for 12 h. Following removal of water by decanting again, crystallization began. The crystals obtained were recrystallized from 300 ml of H₂O/EtOH (½). The crystals were isolated by suction filtration and then dried at 40° C. under reduced pressure.

Yield: 19.7 g (85%) m.p.: 84-87° C.

### 3.2 1,13-bis[(4-Aminophenyl)amino]-4,7,10-trioxatridecane×4 HCl

To 18.5 g of 1,13-bis[(4-nitrophenyl)amino]-4,7,10-trioxatridecane in 400 ml of ethanol there were added 1.5 g of Pd/C (5%). Catalytic reduction was carried out with hydrogen, while shaking. After the end of hydrogen absorption 150 ml of dilute HCl were added, the mixture was filtered, and the filtrate was evaporated to dryness. The resultant oil was admixed with EtOH a number of times and concentrated on a rotary evaporator until crystallization began. The product was subsequently dried at 40° C. under reduced pressure.

Yield: 19.9 g (90%) m.p.: 180-210° C. (decomposition)

### b) Coloration Experiments

The coloration experiments described below were carried out using the hereinabove-described developer components (1), (2), and (3) of the invention.

For the preparation of the colorant, or coloring cream, 50 g of a cream base were weighed out into a 250 ml glass beaker and melted at 80° C. The composition of the cream base used was as follows (amounts in % by weight):

| | |
|---|---|
| 17.0% | Hydrenol ® D¹ |
| 4.0% | Lorol tech.² |
| 40.0% | Texapon ® NSO³ |
| 25.0% | Dehyton ® K⁴ |
| 1.5% | Eumulgin ® B2⁵ |
| 12.5% | Water |

¹C₁₆₋₁₈ fatty alcohol (INCI name: Cetearyl alcohol) (Cognis)
²C₁₂₋₁₈ fatty alcohol (INCI name: Coconut alcohol) (Cognis)
³Lauryl ether sulfate, sodium salt (about 27.5% active substance; INCI name: Sodium Laureth Sulfate) (Cognis)
⁴N,N-Dimethyl-N-(C₈₋₁₈-cocamidopropyl)ammonium acetobetaine (about 30% active substance; INCI name: Aqua (Water), Cocamidopropyl Betaine) (Cognis)
⁵Cetyl stearyl alcohol with about 20 EO units (INCI name: Ceteareth-20) (Cognis)

The substances for investigation were suspended, or dissolved with heating, in distilled water separately, as developers and couplers. In each case 1/400 mol of developer and coupler respectively were used, Subsequently ammonia (<1 ml; 25% strength ammonia solution) was added until the pH was between 9 and 10. Addition of ammonia produced a solution.

The dissolved developers and couplers were incorporated in succession into the hot cream. The formula was subsequently made up to 97 g with distilled water and a pH of 9.5 was set using ammonia. After the batch had been made up to 100 g with distilled water it was stirred cold (<30° C.), forming a homogeneous cream.

The cream was diluted as follows for the different colorations:

25 g cream+25 g distilled water (A)

25 g cream+25 g H₂O₂, 1% (B)

25 g cream+25 g H₂O₂, 9%′ (C)

A lock of hair (80% gray; 330 mg to 370 mg in weight) was placed in each of the mixtures thus obtained. Each of the mixtures and locks of hair was then placed on a watchglass and the locks of hair were embedded thoroughly into the coloring creams. After an exposure time of 30 minutes (±1 minute) at room temperature the locks of hair were removed and washed with Texapon EVR® (Na lauryl ether sulfate) repeatedly until the excess color had been removed. The locks of hair were dried in air and their shade was determined under the daylight lamp (calorimeter HE240A) and recorded (Taschenlexikon der Farben, A. Kornerup and J. H. Wanscher, 3rd, unrevised edition 1981, Muster-Schmidt Verlag; Zurich, Gottingen).

The results obtained in the coloration investigations are set out in Tables I and II below.

TABLE I
Colorations of the developer components of the
invention with standard couplers
(developer/coupler molar ratio = 1/2)

| | Developer: | | |
|---|---|---|---|
| Coupler: | 1 | 2 | 3 |

A Air oxidation

| | | | |
|---|---|---|---|
| Resorcinol | pale | pale | olive-brown |
| 1-Naphthol | n.d. | blue-gray | n.d. |
| 3-Amino-6-methoxy-2- | olive- | n.d. | olive-brown |
| (methylamino)pyridine × 2 | brown | | |
| HCl | | | |
| 5-Amino-2-methylphenol | gray-brown | café-au- | café-au-lait |
| | | lait | |
| 2-Amino-3-hydroxypyridine | pale | hair brown | nougat- |
| | | | colored |
| 2-(2′,4′-Diaminophenoxy)- | n.d. | dark blue | n.d. |
| ethanol × 2 HCl | | | |
| 1,3-bis(2′,4′-Diamino- | dark blue | dark blue | dark blue |
| phenoxy)propane × 4 HCl | | | |
| 2,7-Dihydroxynaphthalene | bamboo- | n.d. | nougat- |
| | yellow | | colored |
| B Oxidation with 1% H₂O₂ | | | |
| Resorcinol | gray-brown | gray-brown | gray-brown |
| 1-Naphthol | n.d. | black-blue | n.d. |
| 3-Amino-6-methoxy-2- | inky-blue | n.d. | violet-gray |
| (methylamino)pyridine × 2 | | | |
| HCl | | | |
| 5-Amino-2-methylphenol | dark | dark | aubergine |
| | purple | purple | |
| 2-Amino-3-hydroxypyridine | violet- | red-brown | dark brown |
| | brown | | |
| 2-(2′, 4′-Diaminophenoxy)- | n.d. | black-blue | n.d. |
| ethanol × 2 HCl | | | |
| 1,3-bis (2′, 4′-Diamino- | inky-blue | black-blue | black-blue |
| phenoxy)propane × 4 HCl | | | |
| 2,7-Dihydroxynaphthalene | earth | n.d. | earth brown |
| | brown | | |
| C Oxidation with 9% H₂O₂ | | | |
| Resorcinol | gray-brown | olive- | olive-brown |
| | | brown | |
| 1-Naphthol | n.d. | black-blue | n.d. |
| 3-Amino-6-methoxy-2- | inky-blue | n.d. | violet-gray |
| (methylamino)pyridine × 2 | | | |
| HCl | | | |
| 5-Amino-2-methylphenol | dark | dark brown | burgundy |
| | purple | | |
| 2-Amino-3-hydroxypyridine | liver- | teak- | eye-brown |
| | brown | colored | |
| 2-(2′,4′-Diaminophenoxy)- | n.d. | black-blue | n.d. |
| ethanol × 2 HCl | | | |
| 1,3-bis (2′,4′-Diamino- | black-blue | black-blue | black-blue |
| phenoxy) propane × 4 HCl | | | |
| 2,7-Dihydroxynaphthalene | olive- | n.d. | olive |
| | brown | | colored |

TABLE II
Colorations of the developer (1) of the
invention and a further developer in a 1/1
ratio and also with standard couplers
(developer/coupler molar ratio = 1/1)

| The following further developer components were employed: | |
|---|---|
| D1 | p-tolylenediamine |
| D2 | p-aminophenol |
| D3 | 2,4,5,6-tetraaminopyrimidine |
| D4 | 4,5-diamino-1-(2′-hydroxyethyl)pyrazole |

| | 2nd Developer: | | | |
|---|---|---|---|---|
| Coupler: | D1 | D2 | D3 | D4 |

A Air oxidation

| | | | | |
|---|---|---|---|---|
| Resorcinol | flax | nougat- | gray- | gray- |
| | yellow | colored | brown | brown |
| 3-Amino-6-methoxy-2- | pale | hair | olive | brown- |
| (methylamino)pyridine × 2 | | brown | | gray |
| HCl | | | | |
| 5-Amino-2-methylphenol | café- | dark | matt | light |
| | au-lait | blond | violet | brown |
| 2-Amino-3-hydroxypyridine | dust | nougat- | brown- | gray- |
| | gray | colored | gray | brown |
| 1,3-bis (2′,4′-Diamino- | dark | blue- | dark | blue- |
| phenoxy)propane × 4 HCl | blue | gray | blue | gray |
| 2,7-Dihydroxynaphthalene | flax | flax | loam- | brown- |
| | yellow | yellow | colored | orange |
| B Oxidation with 1% H₂O₂ | | | | |
| Resorcinol | chocolate- | hair | madeira | violet- |
| | brown | brown | brown | brown |
| 3-Amino-6-methoxy-2- | black- | gray | dark | black- |
| (methylamino)pyridine × 2 | blue | | green | blue |
| HCl | | | | |
| 5-Amino-2-methylphenol | dark | red- | dark | oxblood |
| | magenta | brown | violet | |
| 2-Amino-3-hydroxypyridine | sepia | red- | roe- | port- |
| | | brown | brown | wine |
| | | | | red |
| 1,3-bis(2′,4′-Diamino- | black- | black- | black- | dark |
| phenoxy)propane × 4 HCl | blue | blue | blue | violet |
| 2,7-Dihydroxynaphthalene | silver- | olive- | mustard- | mouse |
| | gray | brown | brown | gray |
| C Oxidation with 9% H₂O₂ | | | | |
| Resorcinol | chocolate- | chest- | gray- | liver- |
| | brown | nut | brown | brown |
| 3-Amino-6-methoxy-2- | black- | gray | dark | black- |
| (methylamino)pyridine × 2 | blue | | green | blue |
| HCl | | | | |
| 5-Amino-2-methylphenol | dark | madeira | dark | oxblood |
| | magenta | brown | violet | |
| 2-Amino-3-hydroxypyridine | dark | brown | bronzino | dark |
| | brown | | | brown |
| 1,3-bis (2′,4′-Diaimino- | black- | black- | black- | dark |
| phenoxy)propane × 4 HCl | blue | blue | blue | violet |
| 2,7-Dihydroxynaphthalene | olive- | olive | olive- | olive |
| | brown | | brown | brown |

The results depicted in Table I and II show that the polyoxaalkyl-bridged p-phenylenediamines (1), (2), and (3) of the invention, and the resultant colorants, exhibit outstanding performance properties. In particular it is possible with the oxidation dyes of the invention, by combination with different couplers and/or with additional developers, to cover a broad spectrum of shades. Consistently outstanding color intensities were achieved.

What is claimed is:

1. Bridged p-phenylenediamines of the general formula (I) 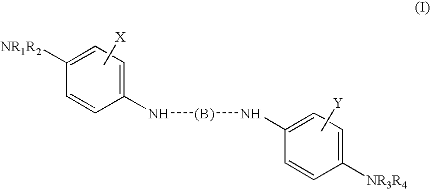
in which
(B) is a polyoxaalkyl bridge having at least two oxygen atoms;
X and Y independently of one another are H, Cl, F or a C₁-C₄ alkyl, hydroxyalkyl, aminoalkyl, alkoxy, C₂-C₄ dihydroxyalkyl, C₁-C₄ monohydroxyalkyl or C₂-C₄ alkenyl group; and
R1, R2, R3, and R4 independently of one another are H or a C₁-C₄ alkyl, C₁-C₄ monohydroxyalkyl or C₂-C₄ dihydroxyalkyl group,
and acid addition salts derived therefrom.

2. The bridged p-phenylenediamines of claim 1, wherein the polyoxaalkyl-bridged p-phenylenediamines have a structure in accordance with formula (II) 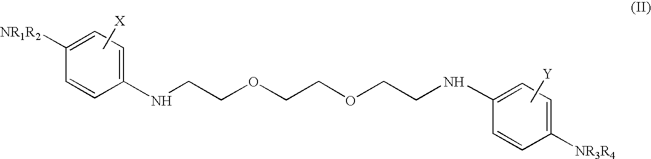
in which
X and Y independently of one another are H, Cl, F or a C₁-C₄ alkyl, aminoalkyl, alkoxy, C₂-C₄ dihydroxy-alkyl, C₁-C₄ monohydroxyalkyl or C₂-C₄ alkenyl group; and
R1, R2, R3, and R4 independently of one another are H or a C₁-C₄ alkyl, C₁-C₄ monohydroxyalkyl or C₂-C₄ dihydroxyalkyl group.

3. The bridged p-phenylenediamines of claim 1, wherein the polyoxaalkyl-bridged p-phenylenediamines have a structure in accordance with formula (III) 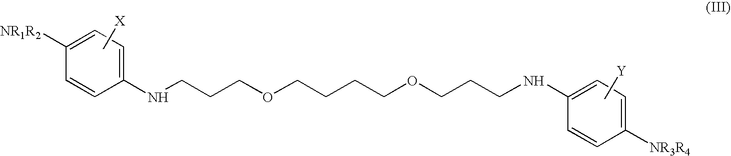
in which
X and Y independently of one another are H, Cl, F or a C₁-C₄ alkyl, aminoalkyl, alkoxy, C₂-C₄ dihydroxy-alkyl, C₁-C₄ monohydroxyalkyl or C₂-C₄ alkenyl group; and
R1, R2, R3, and R4 independently of one another are H or a C₁-C₄ alkyl, C₁-C₄ monohydroxyalkyl or C₂-C₄ dihydroxyalkyl group.

4. The bridged p-phenylenediamines of claim 1, wherein the polyoxaalkyl-bridged p-phenylenediamines have a structure in accordance with formula (IV) 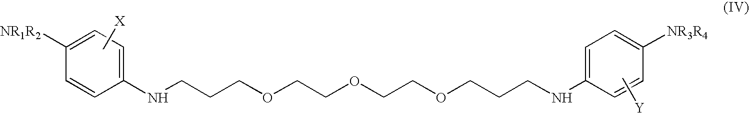
in which
X and Y independently of one another are H, Cl, F or a C₁-C₄ alkyl, aminoalkyl, alkoxy, C₂-C₄ dihydroxy-alkyl, C₁-C₄ monohydroxyalkyl or C₂-C₄ alkenyl group; and
R1, R2, R3, and R4 independently of one another are H or a C₁-C₄ alkyl, C₁-C₄ monohydroxyalkyl or C₂-C₄ dihydroxyalkyl group.

5. The bridged p-phenylenediamines of of claim 1 wherein X and Y and also radicals R1, R2, R3, and R4 are H.

6. A method of using the bridged p-phenylenediamines of claim 1 and/or their water-soluble salts as a developer component in oxidation hair colorants comprising applying the bridged p-phenylenediamine compound onto hair as part of an oxidative hair color treatment.

7. An oxidation colorant composition for coloring keratinic fibers comprising, in a cosmetically acceptable vehicle, at least one polyoxaalkyl-bridged p-phenylenediamine of claim 1 as a developer component.

8. The oxidation colorant of claim 7 further comprising at least one coupler component.

9. The oxidation colorant of claim 8 wherein the coupler component is selected from the group consisting of m-phenylenediamine derivatives, naphthols, resorcinol and resorcinol derivatives, pyrazolones, and m-aminophenol derivatives.

10. The oxidation colorant of claim 9 wherein the coupler component is selected from the group consisting of 1-naphthol, 1,5-, 2,7-, and 1,7-dihydroxynaphthalene, 5-amino-2-methylphenol, m-aminophenol, resorcinol, resorcinol monomethyl ether, m-phenylenediamine, 1-phenyl-3-methylpyrazol-5-one, 2,4-dichloro-3-aminophenol, 1,3-bis(2′,4′-diaminophenoxy)propane, 2-chlororesorcinol, 4-chlororesorcinol, 2-chloro-6-methyl-3-aminophenol, 2-amino-3-hydroxypyridine, 2-methylresorcinol, 5-methylresorcinol, 3,5-diamino-2-methoxytoluene, 5-(β-hydroxyethyl)amino-2-methylphenol, and 2-methyl-4-chloro-5-aminophenol.

11. The oxidation colorant of claim 7 further comprises at least one additional developer component selected from the group consisting of 2,4,5,6-tetraaminopyrimidine, p-aminophenol, N,N-bis (2′-hydroxyethyl)-p-phenylenediamine, 2-(2′,5′-diaminophenyl)ethanol, 2-(2′,5′-diaminophenoxy)ethanol, 4-amino-3-methylphenol, 2-aminomethyl-4-aminophenol, 2-hydroxy-4,5,6-triaminopyrimidine, 2,4-dihydroxy-5,6-diamino-pyrimidine, 2,5,6-triamino-4-hydroxypyrimidine, 1,3-N,N′-bis(2′-hydroxyethyl)-N,N′-bis(4′-aminophenyl)diaminopropan-2-ol, 4-amino-2-((diethylamino)methyl)phenol, bis(2-hydroxy-5-aminophenyl)methane, and 4,5-diamino-1-(2′-hydroxyethylpyrazole) or salts thereof.

12. The oxidation colorant of claim 7 wherein the colorant contains developer components in an amount of from 0.005 to 20% by weight, based on the total colorant.

13. The oxidation colorant of claim 7 further comprising at least one direct dye.

14. A process for preparing the bridged p-phenylenediamines of claim 1 comprising
a) reacting 1-fluoro-4-nitrobenzene with a polyoxa-alkyldiamine, using 1-fluoro-4-nitrobenzene: polyoxaalkyldiamine ratio of 2:1, and
b) hydrogenating the intermediate obtained in step a).

15. The process of claim 14 wherein the reaction of 1-fluoro-4-nitrobenzene with polyoxoalkyldiamine occurs in DMSO at elevated temperatures.

16. The process of claim 15 wherein the temperature is more than 50° C.

17. The process of one of claim 14 wherein the polyoxoalkyldiamine is selected from the group consisting of 2,2′-(ethylenedioxy)diethylamine, 1,4-bis(3-aminopropoxy)butane, and 4,7,10-trioxa-1,13-tridecanediamine.
